(19) United States
(12) Patent Application Publication
(10) Pub. No.: US 2024/0307567 A1
(43) Pub. Date: Sep. 19, 2024

(54) METHOD FOR LEVEL REGULATION OF FEED WATER IN A STEAM STERILIZER, AND STEAM STERILIZER

(71) Applicant: MELAG Medizintechnik GmbH & Co. KG, Berlin (DE)

(72) Inventors: Tobias LICHT, Berlin (DE); Eberhard VON DER WAYDBRINK, Paulinenaue (DE); Elena SCHMALZ, Berlin (DE)

(21) Appl. No.: 18/597,443

(22) Filed: Mar. 6, 2024

(30) Foreign Application Priority Data
Mar. 14, 2023 (EP) 23161848.9

(51) Int. Cl. A61L 2/07; A61L 2/24; F22B 1/28; F22D 5/26

(52) U.S. Cl. CPC A61L 2/07; A61L 2/24; F22D 5/26; A61L 2202/11; A61L 2202/122; A61L 2202/14; F22B 1/285

(57) ABSTRACT

It is provided a method for level regulation of feed water in a chamber of a steam sterilizer or in a steam generator with a chamber of a steam sterilizer connected therewith wherein the feed water is heated by means of a heating element and steam thereby is generated for the chamber. A temperature change rate of the heating element is determined and refeeding with feed water is effected in dependence on the determined temperature change rate of the heating element.

### CROSS REFERENCE TO RELATED APPLICATION

The present application claims priority to European Patent Application No. 23161848.9 filed on Mar. 14, 2023. The entire contents of the above-listed application are hereby incorporated by reference for all purposes.

### TECHNICAL FIELD

This disclosure relates to a method for level regulation of feed water in a chamber of a steam sterilizer or in a steam generator with a chamber of a steam sterilizer connected therewith and to a steam sterilizer.

### BACKGROUND

In steam sterilization, a sterilization is achieved by means of steam. For this purpose, the steam in the sterilization chamber must have a certain temperature-pressure characteristic over a defined time period, in order to ensure that a sterilization of loaded goods present in the sterilization chamber actually is achieved. The steam is generated by evaporation of, in particular deionized, feed water. For this purpose, feed water is heated and evaporated directly in the sterilization chamber or is supplied to the sterilization chamber via an external steam generator. To operate such a steam sterilizer in an energy-efficient way, the amount of feed water in the external steam generator or in the chamber should be chosen optimally. It should be as low as possible, but at the same time sufficient to avoid overheating of the heating element and hence a premature degradation of the same as well as disturbances in operation.

For this purpose, various methods for regulating the feed water level are known from the prior art. The same make use either of the throughflow, the filling level or the temperature in order to ensure a feed water level as optimal as possible over the entire sterilization process.

For example, DE 102 60 895 A1 describes the use of flow sensors for feed water detection. Depending on the operating principle, different components are used here, such as for example measuring turbines, vortex flow or ultrasonic flow sensors, flow monitors or differential pressure sensors. These additional components increase the costs and are an additional source of errors.

The level monitoring described for example in DE 10 2009 044 053 A1 also requires additional components with the corresponding cost disadvantage.

Level monitoring by means of conductivity measurement as described in EP 383 327 A1 cannot be applied to steam sterilization due to the low conductivity of the deionized water used in the sterilization.

The filling level can also be inferred by means of temperature measurement. EP 193 863 A2 for example describes the determination of the filling level by means of a capillary tube regulator. For each filling level, a temperature sensor here is arranged in the vicinity of the associated heating element, which forms part of a switching system filled with an expansion liquid. Here, it is disadvantageous that such systems only permit a fixed temperature switching point and have a hysteresis with respect to falling below the temperature associated with the temperature switching point.

EP 1 108 384 B1 also utilizes the temperature measurement, here by means of a PTC resistor arranged on or in the vicinity of the heating element, for filling level determination.

From DE 60 004 509 T2, for example, a regulation of feeding in dependence on the temperature of the heating element is known. Such temperature-controlled level regulations also involve certain disadvantages. In such temperature-controlled regulations, a feeding operation in general is initiated by exceeding a fixed temperature limit value. Such a regulation involves various disadvantages. First of all, such temperature limit values naturally, in the pressure build-up phase, must lie above the temperature necessary for the pressure build-up phase (e.g. 120° C.) and, in the sterilization phase, even above the sterilization temperature (e.g. 134° C.). Refeeding limited to the case of need only can be ensured in this way. To ensure a fail-safe operation, in particular also under real, not always ideal conditions, it is necessary to choose a higher temperature limit value. Deposits, in particular on a heating element arranged within the steam generator or in the sterilization chamber, for example deteriorate the heat transfer from the heating element to the feed water. This is the case in particular when no deionized water is used as feed water for cost reasons or for lack of availability. According to experience, heating elements incorporated in a chamber also exhibit strong deposits during their operation, consisting of ingredients from care oils for hand and angle pieces in the dental field, care products for surgical instruments and deposits due to non-professional care. In operation, temperature limit values between 160° C. and 200° C. therefore can lead to incorrectly triggered feeding operations. In practice, only distinctly higher limit values of up to 210° C. were found to be expedient. Even with these high limit values, undesired triggering of an overheat protection switch can occur. As the overheat protection switch represents a safety component and separates the heating element from the controller and can be reset again only by manual actuation, any unnecessary triggering of the overheat protection switch should be avoided.

It is also disadvantageous that a high temperature limit value leads to longer and stronger heating of the heating element. Dry heating leads to deposits to an increased extent, for example on a heating element arranged within the steam generator or in the sterilization chamber, so that the susceptibility to failure is aggravated. Moreover, an effective overheat protection cannot be obtained with fixed temperature limit values. Due to a dynamic caused by prolonged response times and correspondingly also prolonged cooling times, overheating of the heating element possibly cannot be prevented despite refeeding. Frequent and strong heating as well as overheating of the heating element, however, disadvantageously shorten its useful life and impair its operability.

### SUMMARY

Against this background it is the object underlying the proposed solution to provide a method for the level regulation of feed water in a chamber of a steam sterilizer or in a steam generator with a chamber of a steam sterilizer connected therewith and a corresponding steam sterilizer, by the use of which the disadvantages known from the prior art are avoided. In particular, it is the object of the solution to provide an inexpensive level regulation which omits additional components, but nevertheless allows a minimization of the amount of feed water and hence of the energy demand and the program runtimes, and effectively prevents regular overheating negative for the useful life of the heating elements and improves the steam quality, in particular by lowering a content of non-condensable gases in the chamber or in the steam generator connected with a chamber, which is achieved in connection with a minimization of the amount of feed water.

This object is achieved by a method for the level regulation of feed water in a chamber of a steam sterilizer or in a steam generator with a chamber of a steam sterilizer connected therewith with features as described herein. Such a method provides that the feed water is heated by means of a heating element and thereby steam is generated for the chamber. According to the solution, the method is characterized by the fact that a temperature change rate of the heating element is determined and refeeding, in particular of the chamber or the steam generator, with feed water is effected in dependence on the determined temperature change rate of the heating element.

This provides for truly demand-oriented feeding. Due to the control based on the change rate, imminent overheating is detected distinctly more quickly as compared to a control on the basis of specified temperature limit values. Thus, it is possible to limit the amount of feed water to the necessary minimum and at the same time avoid constantly recurring overheating of the heating element.

The method of the solution is applied both for the level regulation in steam sterilizers with internal steam generation, in which the steam generation is effected directly in a chamber of the sterilizer itself. The chamber for example is a sterilization chamber for receiving loaded goods to be sterilized by means of steam. In the internal steam generation, the chamber, in particular the sterilization chamber, at the same time also acts as a chamber for generating steam from feed water. For this purpose, a heating element is provided, which for example is arranged in the chamber. In the vicinity of the heating element, a receptacle for feed water can be separated from the rest of the chamber. The method of the solution, however, is likewise applicable to steam sterilizers with external steam generation. Here, the steam is not produced in a chamber, in particular sterilization chamber, of the sterilizer itself, but in an external steam generator, and supplied to the chamber, in particular the sterilization chamber. Sterilization chamber and steam generator hence are different, but in fluid connection with each other. The steam generator can also be referred to as a steam boiler, in particular in the sense of EN 14222.

The level regulation according to the solution is used for example in a fractionated vacuum method. The fractionated vacuum method is a venting method, in which the sterilization conditions are produced via repeated fractionations. In general, two to four fractionations are provided. After an initial pressure build-up and evacuation phase, pressure build-up phases and evacuation phases alternate in order to achieve an increasingly higher air dilution. The control of the pressure build-up and evacuation phases usually is effected by means of specified pressure turning points until reaching a target pressure of the desired sterilization temperature. This is followed by the sterilization phase with regulated temperature, and a pressure release with subsequent drying phase.

Feed water in particular comprises demineralized, i.e. deionized, or distilled water. In accordance with the solution, a temperature change rate refers to a change of the temperature per unit time, for example in K/s. The temperature change rate in one variant is determined as a time derivative of the temperature. The determination of a temperature change rate comprises both the direct measurement of a temperature change rate and the indirect determination of a temperature change rate from measured temperatures and time intervals. For example, the change rate is determined as the difference of a temperature value measured at a first time and a temperature value measured at a second time different from the first time and is divided by the time interval between the first and the second time, and thus the temperature change rate can be determined. In one embodiment, the time interval used for determining the temperature change rate is between 0.1 s and 5 s, in particular 1 s. In an alternative embodiment, the temperature change rate is formed on the basis of more than two temperature values, in particular on the basis of temperature values averaged over a time period. In one variant, the change rate is calculated by means of integral formation. The integral formation suppresses the noise component of the temperature measurement. For determining the temperature change rate a temperature sensor for example is provided in the steam sterilizer, in particular at the heating element.

When refeeding, feed water is introduced into the volume heated by the heating element, i.e. into the chamber or the external steam generator.

In one variant, the start of refeeding is triggered in dependence on the determined temperature change rate of the heating element. This provides for rapid action against imminent overheating and for avoiding unnecessary heating of the heating element. While in the case of a temperature-controlled regulation with a temperature limit value of 210° C. and a desired need of refeeding at e.g. 100° C. an unnecessary heating of the heating element by 110 K cannot be prevented, a change-rate-controlled regulation, which is triggered by a determined temperature change rate, typically only leads to an (actually) superfluous heating by 10 K to 20 K and only in exceptional cases by up to 30 K.

In one variant, the start of refeeding is triggered when the determined temperature change rate exceeds a specified switch-on change rate. The needs-based refeeding thus is started when the determined temperature change rate exceeds a specified switch-on change rate. In one variant, the specified switch-on change rate comprises a fixed switch-on value. For example, the fixed switch-on value is between 0.5 K/s and 5 K/s, in particular 2 K/s.

In one variant, the end of refeeding is triggered in dependence on the determined temperature change rate of the heating element. Alternatively or additionally, the end of refeeding is triggered in dependence on a determined pressure change rate, wherein the pressure change rate is a pressure change rate determined in the chamber. The end of needs-based refeeding thus is controlled via the determined temperature change rate or via the determined pressure change rate. Refeeding can thus be limited to a reduced duration. The amount of feed water thus is kept as low as possible, while at the same time avoiding imminent overheating of the heating element.

In one variant, the end of refeeding is triggered when the determined temperature change rate of the heating element falls below a specified first switch-off change rate of the temperature. In one variant, the specified first switch-off change rate of the temperature comprises a fixed switch-off value of the temperature. For example, the fixed switch-off value of the temperature is between −5 K/s and 2 K/s, in particular 1 K/s. Alternatively or additionally, the end of refeeding is triggered when the determined pressure change rate falls below or, in a further variant, exceeds a specified switch-off change rate of the pressure. In one embodiment, the fed amount of feed water additionally can be inferred from the determined pressure change rate with known power consumption of the heating element. In one variant, the specified switch-off change rate of the pressure comprises a fixed switch-off value of the pressure. For example, the fixed switch-off value of the pressure is between-5 mbar/s and +5 mbar/s, in particular 0 mbar/s.

In one variant, on triggering the end of refeeding, refeeding is continued for a specified additional refeeding period and is terminated only at the end of the specified additional refeeding period. The specified additional refeeding period can be determined for example by a specified time interval, in particular it can be effected in a time-controlled way, or be determined in dependence on the time period between the triggering of the start of refeeding and the triggering of the end of refeeding. The determination of the additional refeeding period in dependence on the triggering of the start and the end of refeeding increases the robustness of the method. In particular, this allows to take account of and compensate reduced flow rates and thus ensure a specified refeeding volume. The end of triggering can have been effected in dependence on the determined temperature change rate or in dependence on the determined pressure change rate.

In one variant, refeeding is effected in a pressure build-up phase of a sterilization process, in particular during a pressure build-up phase of a fractionated vacuum method. During the pressure build-up phase refeeding can be effected once or several times. A pressure build-up phase ends for example when a fixed target pressure is reached.

In one embodiment, the switch-on change rate is specified in dependence on other control variables, for example a measured temperature of the heating element or a measured pressure in the chamber. For example, different values for the switch-on change rate can be specified for different temperature or pressure ranges. For a first temperature range a first switch-on change rate can be specified, for a second temperature range a second switch-on change rate, and for a third temperature range a third switch-on change rate. The first temperature range for example comprises temperatures below 80° C., the second temperature range temperatures between 80° C. and 120° C., and the third temperature range temperatures between 121° C. and 134° C. In an alternative variant, the specified switch-on change rate is a function of the temperature, wherein the function in particular is chosen such that it corresponds to a typical course of a temperature curve of the heating element. In one embodiment, the specified first switch-off change rate of the temperature is dependent on the specified switch-on change rate. For example, the first switch-off change rate of the temperature is between 0 K/s and 5 K/s, in particular 1 K/s, lower than the specified switch-on change rate.

In one variant, the temperature change rate of the heating element is determined continuously. The temperature change rate is determined for example every second. In one variant, the continuous determination of the temperature change rate starts when a specified temperature of the heating element is reached. This specified temperature for example is between 40° C. and 120° C., in particular 80° C. This means that from a specified temperature of the heating element change rate monitoring is started. This provides for a robust regulation process.

In one variant, the heating element is switched off when an upper limit temperature of the heating element is exceeded. This upper limit temperature for example lies between 120° C. and 240° C., in particular at 180° C. Alternatively, the heating element is switched off when the determined temperature change rate of the heating element exceeds a second switch-off change rate of the temperature. The second switch-off change rate of the temperature for example lies between 5 K/s and 12 K/s, in particular at 6 K/s. Feeding, however, is continued continuously or discontinuously. The detection of insufficient or completely missing feed water, for example due to a defective or worn feed pump or an empty feed water tank, is possible herewith. Overheating of the heating element also is prevented in this case of failure and prolongs the useful life of the heating element and hence also of the associated steam sterilizer.

In one variant, the heating element is switched on, in particular after switch-off on exceedance of an upper limit temperature or on exceedance of the second switch-off change rate of the temperature, when the temperature falls below a lower limit temperature. This lower limit temperature for example is between 0 K and 80 K, in particular 10 K, below the upper limit temperature. In one variant, it is provided that on renewed exceedance of the upper limit temperature or on renewed exceedance of the second switch-off change rate of the temperature, the method is stopped directly, in particular heating and feeding is terminated. What is stopped in particular is a sterilization method during which the level regulation takes place. Alternatively, the steps of switching on the heating element when the temperature falls below the lower limit temperature and switching off the heating element on exceedance of the upper limit temperature or on exceedance of the second switch-off change rate of the temperature are repeated once or several times, and the method then is stopped, in particular heating and feeding is terminated. What is stopped in particular is the sterilization method during which the level regulation takes place. This ensures that in the case of an actual lack of feed water, which cannot be remedied during the regulation, or in the case of a complete lack of feed water, the sterilization method is terminated.

In one variant, initial feeding of feed water into the chamber or into the steam generator is started when during a first evacuation phase of the chamber the pressure in the chamber falls below a specified pressure. This initial feeding can be effected for example in a time-controlled way. The time-controlled initial feeding ensures that a sufficient minimum amount of feed water is available, in particular at the beginning of a sterilization method and before a pressure build-up phase. This is important to prevent overheating of the heating element. Initial feeding, in particular during a first evacuation phase of the chamber, can however also be regulated via the temperature of the heating element and the determined temperature change rate. In one variant, initial feeding of feed water into the chamber or into the steam generator, in particular during a first evacuation phase of the chamber, is started when the heating element reaches a specified control temperature. For example, the specified control temperature is between 40° C. and 100° C., in particular 80° C. Initial feeding is terminated for example when the determined temperature change rate has reached a specified third switch-off change rate of the temperature. In this way, a minimum amount of feed water can also be ensured and overheating of the heating element can thus be prevented.

The object underlying the solution also is achieved by a steam sterilizer having features as described herein. Such a steam sterilizer includes a heating element for heating feed water. The feed water provided for heating is located directly in a chamber of the steam sterilizer or in a steam generator of the steam sterilizer provided for external steam generation. The steam generator is connected with a chamber of the steam sterilizer so that steam generated in the steam generator can get into the chamber.

The steam sterilizer of the solution is characterized in that it is adapted to carry out a method with the features as described herein in operation.

Thus, there is provided a steam sterilizer which provides for truly needs-based feeding and hence for an energy-efficient operation. The steam sterilizer is less susceptible to failure and has a longer useful life, as the useful life of the heating element is prolonged by the smaller number of overheatings.

As described with respect to the method of the solution, the steam sterilizer of the solution comprises sterilizers with internal steam generation and sterilizers with external steam generation. The heating element can be arranged within the chamber or the steam generator, in particular in the region of the floor, in a wall of the chamber or the steam generator, or outside the chamber or the steam generator, but in contact with the chamber or the steam generator.

In one variant, the heating element is configured as a tubular radiator and arranged on the floor of the chamber of the steam sterilizer with internal steam generation.

The steam sterilizer in one variant includes a temperature sensor for determining a temperature change rate. The temperature sensor for example is arranged such that it detects the point of the strongest change in temperature. In one variant, the heating element is arranged on the floor of the chamber or the steam generator, and the temperature sensor is arranged on an upper side of the heating element. Upper side refers to the side of the heating element facing away from the floor and getting dry first on lowering of the feed water level. The response time thereby is shortened.

In one variant, the steam sterilizer comprises software which, when executed on a processor of the steam sterilizer, makes the processor carry out the method having features as described herein. In particular, the software realizes a computer-implemented method with features as described herein. This implementation represents an implementation of the method of the solution which is to be carried out and retrofitted easily and is inexpensive.

All variants and embodiments of the method can be combined with each other in any way and can be transferred to the steam sterilizer either individually or in any combination. In the same way, all variants and embodiments of the steam sterilizer can be combined with each other in any way and can be transferred to the method individually or in any combination.

### BRIEF DESCRIPTION OF THE DRAWINGS

Details of aspects of the solution claimed here will be explained in detail below with reference to exemplary embodiments and Figures.

FIG. 1 shows a representation of the determination of the temperature change rate according to an embodiment of the solution.

FIG. 2 shows a representation of the switch-on and switch-off change rates according to an embodiment of the solution.

FIG. 3 shows a representation of a method according to an embodiment, in particular of a refeeding of the solution started and ended by means of a temperature change rate.

FIG. 4 shows a representation of a method according to an embodiment, in particular of a change-rate-controlled feeding in pressure build-up phases of the solution.

FIG. 5 shows a representation of a steam sterilizer according to an embodiment of the solution.

FIG. 6 shows a heating element with temperature sensor according to an embodiment of the solution.

### DETAILED DESCRIPTION

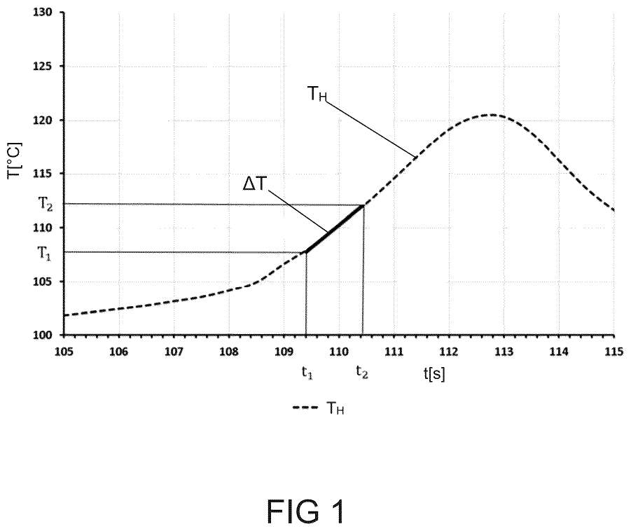

FIG. 1 illustrates the determination of a temperature change rate ΔT, i.e. of a change of the temperature T_{H} of the heating element 4 per unit time, of a heating element 4 of a steam sterilizer 1 according to an embodiment of the solution. The temperature change rate ΔT is determined as a measured change of the temperature T_{H} of the heating element 4 per unit time. It hence corresponds to the slope of the temperature profile curve T_{H} (t). At the time tᵢ, the determined temperature change rate ΔT in the present embodiment is given by:

$$Δ T ( {t}_{i} ) = \frac{T ( {t}_{i} ) - T ( {t}_{i - 1} )}{{t}_{i} - {t}_{i - 1}} .$$

The change rate ΔT is determined continuously at given time intervals, for example every second. To achieve a continuous course of the temperature change rate ΔT, the temperature change rate ΔT can also be determined continuously. Averaging should be chosen such that the dynamic underlying the sterilization process remains visible and is not suppressed.

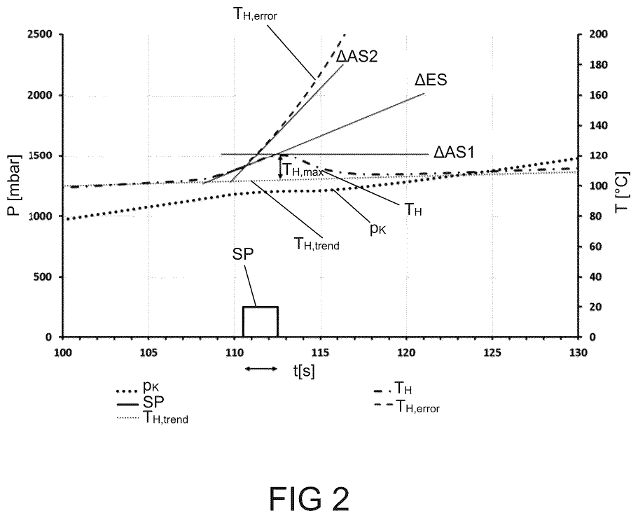

FIG. 2 shows a level regulation for feed water in a chamber 2 or in a steam generator of a steam sterilizer connected with a chamber 2 according to an embodiment. FIG. 2 shows the temporal course of the temperature T_{H} of the heating element 4 and the temporal course of the pressure p_{K} in the chamber 2. The temperature change rate ΔT of the heating element 4 is determined as described for example with respect to FIG. 1. Refeeding of feed water then is effected in dependence on the determined temperature change rate ΔT. In the case of an external steam generation the feed water is fed into the external steam generator, and in the case of an internal steam generation it is fed directly into the chamber 2. For example, the start of refeeding is triggered in dependence on the temperature change rate ΔT. This provides for truly needs-based refeeding, in particular for a fast response to temperature deviations. For example, in the present embodiment a switch-on change rate ΔES is specified. Refeeding is started when the determined temperature change rate ΔT exceeds the specified switch-on change rate ΔES. The specified switch-on change rate ΔES for example comprises a fixed value. This value for example lies between 0.5 K/s and 5 K/s, in particular at 2 K/s. When the temperature profile T_{H} deviates from the trend line T_{H,trend} of the temperature profile, overheating of the heating element 4 is imminent. Because refeeding is started in dependence on the determined temperature change rate ΔT, this overheating is intercepted. The rise in temperature flattens out. Refeeding for example is also terminated in dependence on the determined temperature change rate ΔT. For this purpose, for example a first switch-off change rate ΔAS1 of the temperature is specified, below which refeeding is terminated. The first specified switch-off change rate ΔAS1 of the temperature in the present case comprises a fixed value, for example between-5 K/s and 2 K/s, in particular 1 K/s. A first specified switch-off change rate ΔAS1 of 0 K/s effects a termination of refeeding at an extreme value, in the present case a maximum T_{H,max}, of the temperature profile T_{H}. Thus, refeeding is terminated as soon as the temperature T_{H} changes from a temperature rise to a temperature drop. The amount of feed water supplied on refeeding thus is minimized. As a result, energy is saved and program runtimes are shortened. In the illustrated diagram, the refeeding operation can be derived from the switching state SP of the feed pump 7.

To provide for refeeding in dependence on the determined temperature change rate ΔT, the temperature gradient ΔT is determined continuously, i.e. the temperature change rate ΔT is monitored continuously. Monitoring the temperature change rate ΔT for example starts as soon as a specified temperature of the heating element 4 of for example between 40° C. and 120°, in particular 80° C., is reached. Thus, a robust regulation process is provided.

In one embodiment (not shown) refeeding is prolonged by a specified additional refeeding period. This means that when the end of refeeding is triggered, refeeding is not terminated immediately, but in addition is also continued for the specified additional refeeding period. Refeeding is terminated only at the end of the additional refeeding period. This ensures a sufficient amount of feed water at the end of the refeeding operation. The additional refeeding period is determined for example by a specified time interval (time-controlled additional refeeding). Alternatively, the additional refeeding period is determined in dependence on the time period between the triggering of the start of refeeding and the triggering of the end of refeeding.

When refeeding is not possible at all or only incompletely due to a case of fault, for example a worn or defective feed pump 7 or an empty feed water tank, overheating of the heating element 4 cannot be prevented by refeeding, as can be derived from the temperature profile T_{H,error} for the case of fault. For this case, a second switch-off change rate ΔAS2 of the temperature is provided, on exceedance of which the heating element 4 is switched off. The second switch-off change rate ΔAS2 of the temperature for example is between 5 K/s and 12 K/s, in particular 6 K/s. Feeding, however, here is continued continuously or discontinuously. In this way, an insufficient amount of feed water is detected and overheating of the heating element 4 in a case of fault can be prevented. Alternatively, an upper limit temperature (not shown) can also be provided instead of a second switch-off change rate ΔAS2, so that the heating element 4 is switched off as soon as the upper limit temperature is exceeded. For example, the upper limit temperature lies between 120° C. and 240° C., in particular at 180° C. After a switch-off of the heating element, due to exceedance of the second switch-off change rate ΔAS2 of the temperature or due to exceedance of the upper limit temperature, the heating element 4 is switched on again as soon as the temperature falls below a lower limit temperature (not shown). This lower limit temperature for example lies between 0 K and 80 K, in particular at 10 K, lower than the upper limit temperature. When the upper limit temperature or the second switch-off change rate ΔAS2 of the temperature then is again exceeded, the sterilization method, in particular heating and feeding, is stopped. Alternatively, the steps of switching on when the temperature falls below the lower limit temperature and switching off when the upper limit temperature is exceeded are repeated once or several times, and the sterilization method then is stopped, in particular heating and feeding is terminated. This ensures a program termination when a case of fault cannot be remedied.

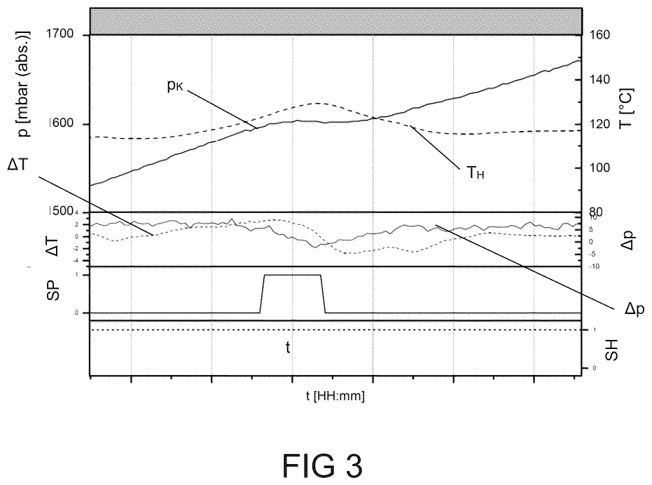

FIG. 3 illustrates a method for the level regulation of feed water according to another embodiment. The method steps explained with respect to FIGS. 1 and 2 are applied. Beside the temperature profile T_{H} and the pressure profile p_{K} there is also shown the temporal course of the determined temperature change rate ΔT and the temporal course of the pressure change rate Δp in the chamber 2 during a pressure build-up phase of a sterilization process, in particular of a fractionated vacuum method. Correspondingly, the pressure p_{K} rises; the determined pressure gradient Δp is positive. The temperature T_{H} of the heating element 4 is chosen such that feed water present in a chamber 2 or in a steam generator of a steam sterilizer 1 is evaporated. Correspondingly, the determined temperature change rate ΔT is almost constant. In the case of a change in temperature at the heating element 4 due to an insufficient amount of feed water, the determined temperature change rate ΔT deviates from zero. When the determined temperature change rate ΔT exceeds a specified switch-on change rate ΔES, refeeding is started. The temperature increase is intercepted. The temperature curve T_{H} flattens out. Correspondingly, the determined temperature change rate ΔT decreases. When the temperature falls below a specified first switch-off change rate ΔAS1 of the temperature, refeeding is terminated. The temperature T_{H} returns to its setpoint value (here 100° C.). The switching state SP of the feed pump 7 shows the beginning and end of refeeding, triggered by the determined temperature change rate ΔT.

As can be derived from the illustrated pressure curve p_{K}, the temporal pressure profile has a maximum. When there is not enough feed water, the pressure p_{K} in the chamber 2 cannot rise any more. When refeeding then is initiated in dependence on the determined temperature change rate ΔT, the newly introduced water must first be heated, before it starts to evaporate. Only then, the pressure p_{K} starts to rise again. Thus, in one embodiment, refeeding can also be effected in dependence on a determined pressure change rate Δp. The determined pressure change rate Δp corresponds to a measured change in pressure in the chamber 2 per time interval. The determined pressure change rate Δp can also be averaged in order to obtain a continuous course. When the determined pressure change rate Δp for example falls below a specified switch-off change rate ΔASp of the pressure, refeeding is terminated. The specified switch-off change rate ΔASp of the pressure for example is between-5 mbar/s and +5 mbar/s, in particular 0 mbar/s. This creates an alternative criterion for terminating the refeeding operation.

The heating element 4 remains switched on during the entire operation, as can be derived from the switching state SH of the heating element 4.

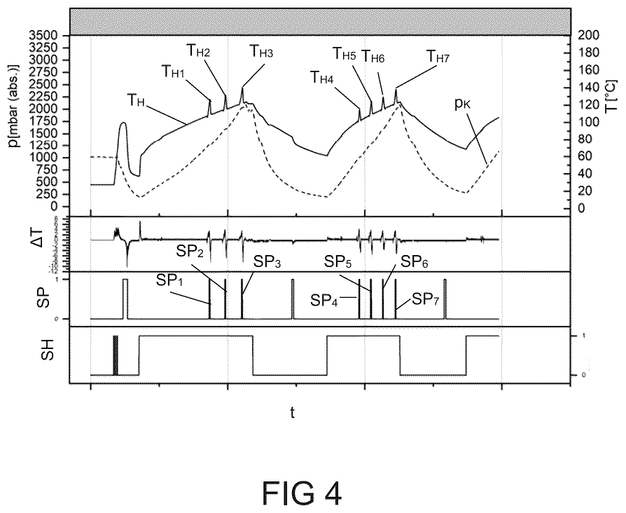

FIG. 4 shows the level regulation according to one embodiment for a plurality of pressure build-up and evacuation phases of a fractionated vacuum method. The method steps explained with respect to FIGS. 1 to 3 are applied. As shown, the sterilization conditions (pressure and temperature in the chamber 2) are reached over a plurality of alternating pressure build-up and evacuation phases, i.e. fractionations. Here, two fractionations are shown, i.e. two pressure build-up phases each followed by an evacuation phase. During the pressure build-up phase the pressure p_{K} in the chamber 2 is built up in particular by evaporation of feed water (supplied with an external steam generator). In the following evacuation phase, the steam is drained by pressure release. The change-rate-based refeeding in one embodiment is effected only during the pressure build-up phases. What is clearly visible are the three temperature peaks T_{H1}, T_{H2}, T_{H3} at the end of the first pressure build-up phase and the four temperature peaks T_{H4}, T_{H5}, T_{H6}, T_{H7} at the end of the second pressure build-up phase. The corresponding increase of the temperature T_{H} of the heating element 4 is detected via the determined temperature change rate ΔT: when, as described with respect to FIGS. 2 and 3, the same exceeds a specified switch-on change rate ΔES, a refeeding operation is triggered. The same is terminated when the determined temperature change rate ΔT again falls below a specified first switch-off change rate ΔAS1 of the temperature. The refeeding operation in addition can be continued for an additional refeeding period. The corresponding refeeding operations SP₁, SP₂, SP₃ of the first pressure build-up phase and the corresponding refeeding operations SP₄, SP₅, SP₆, SP₇ of the second pressure build-up phase can be derived from the switching state of the feed pump. In addition, as shown in FIG. 4, process-related time-controlled refeeding operations can be effected, which will not be discussed here in more detail. The heating element 4 remains switched on during the pressure build-up phase, if no case of fault occurs. During the evacuation phases the heating element 4 is switched off. Switching off and switching on the heating element 4 between the individual pressure build-up and evacuation phases generally is effected via pressure switching points. The heating phases can be derived from the switching state of the heating element 4.

It is important to provide a minimum amount of feed water at the beginning of a sterilization operation and before a pressure build-up phase, in order to prevent overheating of the heating element 4. For this purpose, various regulations of an initial feeding are possible. When the sterilization method starts with an evacuation phase, i.e. when the first phase is an evacuation phase, an initial feeding is effected in a time-controlled way when the pressure in the chamber 2 falls below a specified pressure. A successful initial feeding only can be detected in the following pressure build-up phase due to the fact that overheating of the heating element 4 does not occur right at the beginning of this phase. Alternatively, the initial feeding is effected when the heating element 4 reaches a specified control temperature, wherein the specified control temperature is between 40° C. and 100° C., in particular 80° C. The initial feeding is terminated when the determined temperature change rate ΔT reaches a specified third switch-off change rate ΔAS3 of the temperature. The third switch-off change rate ΔAS3 of the temperature in one embodiment is identical with the first switch-off change rate ΔAS1 of the temperature. The method for initial feeding ensures a minimum amount of feed water at the beginning of a sterilization method, in particular of a fractionated vacuum method.

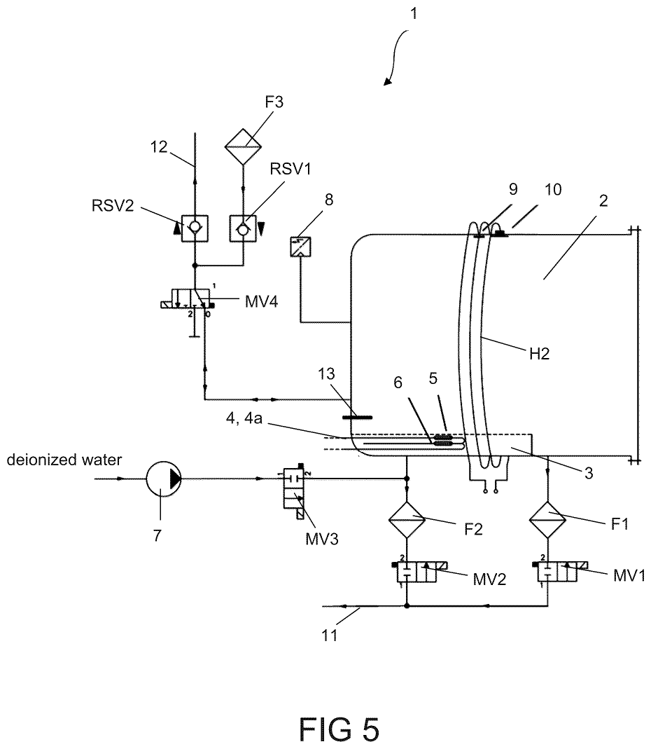

FIG. 5 by way of example shows a steam sterilizer 1 according to an embodiment of the solution. The steam sterilizer 1 is characterized in that it is suited and adapted to carry out a method for level regulation in operation as explained with respect to the preceding FIGS. 1 to 4. For this purpose, the steam sterilizer 1 for example includes a software which, when executed on a processor, causes the same to carry out a method for level regulation as explained with respect to the preceding FIGS. 1 to 4.

The steam sterilizer 1 includes a heating element 4 for heating feed water present in a chamber 2 of the steam sterilizer 1 or of a steam generator of the steam sterilizer 1 connected with a chamber 2 of the steam sterilizer 1.

In terms of construction, the steam sterilizer 1 can provide both an external and an internal steam generation. In the present case, a steam sterilizer 1 is shown with an internal steam generation. This means that feed water is evaporated directly in a chamber 2 of the sterilizer 1 and is used there for sterilizing loaded goods present in the chamber 2. For this purpose, the heating element 4 is arranged for example in the chamber 2, here in the region 3 of a floor of the chamber 2. The heating element 4 is configured for example as a tubular radiator 4a. By means of a temperature sensor 5 mounted directly on the tubular radiator 4a, the temperature change rate ΔT is determined continuously. In one variant, a safety temperature limiter 6 is additionally arranged on the tubular radiator 4a, which separates the tubular radiator 4a from a voltage supply in the case of a defective controller. In one embodiment the chamber 2, in particular its floor, is inclined with respect to a horizontal so that only the front part of the tubular radiator 4a is exposed in the case of a lack of feed water.

During an evacuation phase, the chamber 2 is evacuated by means of a vacuum pump, possibly with an upstream steam condenser, (both not shown) and by simultaneously opening the solenoid valve MV1. Furthermore, a filter F1 is provided in one embodiment in order to protect the solenoid valve MV1 from impurities, for example due to detached deposits from the chamber. Feeding is effected with a feed pump 7 and simultaneous opening of the solenoid valve MV3 from below into the region 3 of the chamber 2 in which the tubular radiator 4a also is arranged.

The region 3 of the tubular radiator 4a is isolated from the remaining floor of the chamber 2. The required feed water thereby is minimized already. The steam exit towards the top is ensured by openings, for example in the sheet isolating the region 3. When too much feed water gets into the isolated region 3, for example after a power failure, excess water runs over the bulkhead into a front region of the chamber 2 during feeding in the sterilization method which continues or follows after the power failure, in which region condensate can also collect. The same can also be pumped off during a succeeding evacuation.

In a pressure build-up phase, pressure is built up by switching on the tubular radiator 4a. A jacket heating H2 arranged on the circumference of the chamber 2 in addition preheats the chamber 2 and ensures a uniform temperature distribution in the chamber 2 as well as a reduction of condensate produced in the chamber 2. The regulation of the jacket heating H2 here is effected via a temperature sensor 9 of the jacket heating H2. There can also be provided a safety temperature limiter 10 for the jacket heating H2.

A fractionated vacuum method carried out in the steam sterilizer 1 can be controlled via a pressure sensor 8 by utilizing an upper and a lower pressure turning point of the fractionations as a trigger for the beginning of the evacuation phase and the pressure build-up phase, respectively. In the chamber 2 at least one temperature sensor 13 of the chamber 2 is provided, which verifies the sterilization temperature and/or can be used for controlling the method.

The pressure release and hence also the release of remaining feed water and condensate at the end of the sterilization is effected by means of a solenoid valve MV2, optionally via a further solenoid valve MV1, in the present case with upstream filter F2 or F1, respectively.

In the subsequent drying phase, drying is effected by means of vacuum. Condensate produced is pumped off via the vacuum pump. After completion of the drying phase, the chamber 2 is aerated by opening a solenoid valve MV4 and sucking in sterile air via a sterile filter due to the negative pressure present in the chamber 2. A check valve RSV1 prevents an ingress of moisture from the chamber 2 into the sterile filter F3.

In the case of a power failure an automatic pressure release (emergency release) is effected via the solenoid valve MV4 into a waste water tank (not shown). The check valve RSV2 prevents non-sterile air from getting into the chamber 2.

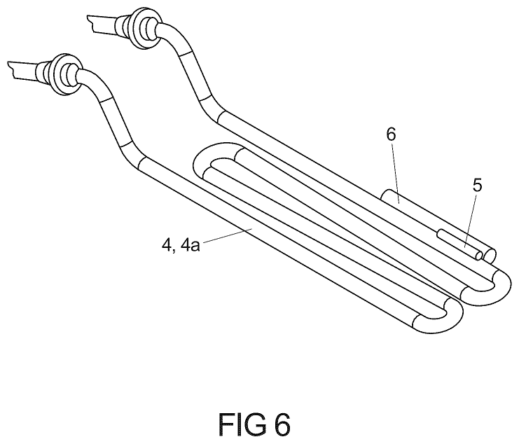

FIG. 6 shows a heating element 4 for heating feed water present in a chamber 2 of a steam sterilizer 1, for example of FIG. 5, or present in a steam generator of a steam sterilizer 1 connected with a chamber 2. In the present case, the heating element 4 is configured as a tubular radiator 4a. On the tubular radiator 4a, a temperature sensor 5 is arranged. By means of the temperature sensor 5 a temperature change rate ΔT is determined and feed water is refed in dependence on the same, as described with respect to FIGS. 1 to 4. For example, the temperature sensor 5 is arranged on an upper side of the tubular radiator 4a, so that the temperature sensor 5 is located in a region of the tubular radiator 4a heated first when the feed water level is too low. In one variant, the chamber 2 additionally is inclined with respect to a horizontal so that only the front part of the tubular radiator 4a is exposed in the case of a lack of feed water. A safety temperature limiter 6 likewise is arranged on the tubular radiator 4a. It serves as an additional protection means and separates the tubular radiator 4a from a voltage supply in the case of a defective controller.

### LIST OF REFERENCE NUMERALS

1 steam sterilizer
2 chamber
3 region
4 heating element
4a tubular radiator
5 temperature sensor
6 safety temperature limiter
7 feed pump
8 pressure sensor
9 temperature sensor of jacket heating
10 safety temperature limiter of jacket heating
11 supply line of vacuum pump
12 supply line of waste water tank
13 temperature sensor of chamber
F1 filter
F2 filter
F3 sterile filter
MV1 solenoid valve
MV2 solenoid valve
MV3 solenoid valve
MV4 solenoid valve
RSV1 check valve
RSV2 check valve
H2 jacket heating
ΔES switch-on change rate
ΔAS1 first switch-off change rate of temperature
ΔAS2 second switch-off change rate of temperature
ΔASp switch-off change rate of pressure
p pressure (absolute)
T temperature
t time
tᵢ time i
T_{H}(tᵢ) temperature of heating element at the time tᵢ
T_{H,trend} temperature of heating element trend line
T_{H,error} temperature of heating element in case of fault
T_{H} temperature of heating element
p_{K} pressure in the chamber (internal or external steam generation)
p(t) temporal pressure profile
Δp determined pressure change rate
ΔT determined temperature change rate
T_{H,max} temperature maximum
SP switching state of feed pump
SH switching state of heating element

1. A method for level regulation of feed water in a chamber of a steam sterilizer or in a steam generator with a chamber of a steam sterilizer connected therewith, wherein the feed water is heated by means of a heating element and steam thereby is generated for the chamber, wherein a temperature change rate of the heating element is determined and a refeeding with feed water is effected in dependence on the determined temperature change rate of the heating element.

2. The method according to claim 1, wherein the start of refeeding is triggered in dependence on the determined temperature change rate of the heating element.

3. The method according to claim 1, wherein the start of the refeeding is triggered when the determined temperature change rate exceeds a specified switch-on change rate.

4. The method according to claim 3, wherein the specified switch-on change rate comprises a fixed switch-on value, wherein the fixed switch-on value is between 0.5 K/s and 5 K/s.

5. The method according to claim 1, wherein the end of the refeeding is triggered in dependence on the determined temperature change rate of the heating element and/or in dependence on a determined pressure change rate, wherein the pressure change rate is a pressure change rate determined in the chamber.

6. The method according to claim 5, wherein the end of the refeeding is triggered when the determined temperature change rate of the heating element falls below a specified first switch-off change rate of the temperature, wherein the specified first switch-off change rate of the temperature comprises a fixed switch-off value of the temperature, wherein the fixed switch-off value of the temperature is between −5 K/s and 2 K/s.

7. The method according to claim 5, wherein the end of the refeeding is triggered when the determined pressure change rate falls below a specified switch-off change rate of the pressure or the end of the refeeding is triggered when the determined pressure change rate exceeds a specified switch-off change rate of the pressure, wherein the specified switch-off change rate of the pressure comprises a fixed switch-off value of the pressure, wherein the fixed switch-off value of the pressure is between −5 mbar/s and +5 mbar/s.

8. The method according to claim 1, wherein, when the end of the refeeding is triggered, the refeeding is continued for a specified additional refeeding period and is terminated only at the end of the specified additional refeeding period, wherein the specified additional refeeding period is determined by a specified time interval or is determined in dependence on the time period between the triggering of the start of the refeeding and the triggering of the end of the refeeding.

9. The method according to claim 1, wherein the temperature change rate of the heating element is determined continuously, wherein the continuous determination of the temperature change rate starts when a specified temperature of the heating element is reached, wherein the specified temperature is between 40° C. and 120° C.

10. The method according to claim 1, wherein the heating element is switched off when an upper limit temperature of the heating element is exceeded, wherein the upper limit temperature is between 120° C. and 240° C., or when the determined temperature change rate exceeds a second switch-off change rate of the temperature, wherein the second switch-off change rate of the temperature is between 5 K/s and 12 K/s, wherein feeding is continued continuously or discontinuously.

11. The method according to claim 10, wherein the heating element is switched on when the temperature falls below a lower limit temperature, wherein the lower limit temperature lies between 0 K and 80 K, below the upper limit temperature.

12. The method according to claim 11, wherein on renewed exceedance of the upper limit temperature or on renewed exceedance of the second switch-off change rate of the temperature the method is stopped directly, in particular heating and feeding is terminated directly, or the steps of switching on the heating element when the temperature falls below the lower limit temperature and switching off the heating element on exceedance of the upper limit temperature or exceedance of the second switch-off change rate of the temperature are repeated once or several times, and the method then is stopped, in particular heating and feeding is terminated directly.

13. The method according to claim 1, wherein an initial feeding of feed water into the chamber or into the steam generator is started when during a first evacuation phase of the chamber the pressure in the chamber falls below a specified pressure and/or when the heating element reaches a specified control temperature, wherein the specified control temperature is between 40° C. and 100° C., and wherein the initial feeding is terminated when the determined temperature change rate reaches a specified third switch-off change rate of the temperature.

14. A steam sterilizer comprising a chamber for receiving feed water or comprising a steam generator connected with a chamber for receiving feed water, wherein the steam sterilizer includes a heating element for heating the feed water, wherein the steam sterilizer is provided and adapted to carry out the method according to claim 1 in operation.

15. The steam sterilizer according to claim 14, wherein the steam sterilizer comprises software which, when executed on a processor of the steam sterilizer, causes the processor to carry out a method for level regulation of feed water in a chamber of a steam sterilizer or in a steam generator with a chamber of a steam sterilizer connected therewith, wherein the feed water is heated by means of a heating element and steam thereby is generated for the chamber, wherein a temperature change rate of the heating element is determined and a refeeding with feed water is effected in dependence on the determined temperature change rate of the heating element.
